SEQUENCE LISTING (1 sequence)

SEQ ID NO: 1 - protein, 140 aa, Artificial Sequence
  Actimmune(r); Interferon gamma 1b
MQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGR

(19) United States
(12) Patent Application Publication
(10) Pub. No.: US 2025/0032584 A1
(43) Pub. Date: Jan. 30, 2025

(54) INHALED INTERFERONS FOR VIRAL RESPIRATORY INFECTIONS

(71) Applicant: THE RESEARCH FOUNDATION FOR THE STATE UNIVERSITY OF NEW YORK, Albany, NY (US)

(72) Inventors: Gerald SMALDONE, Stony Brook, NY (US); Rany CONDOS, New York, NY (US)

(21) Appl. No.: 17/907,213

(22) PCT Filed: Mar. 24, 2021
(86) PCT No.: PCT/US2021/024013

§ 371 Date: Sep. 23, 2022

Related U.S. Application Data
(60) Provisional application No. 63/000,469, filed on Mar. 26, 2020.
(60) Provisional application No. 63/000,525, filed on Mar. 27, 2020.
(60) Provisional application No. 63/056,505, filed on Jul. 24, 2020.

(51) Int. Cl. A61K 38/21; A61K 9/00; A61K 47/12; A61K 47/26; A61P 31/14

(52) U.S. Cl. CPC A61K 38/217; A61K 9/0078; A61K 47/12; A61K 47/26; A61P 31/14

(57) ABSTRACT

Aerosolized interferon-γ is disclosed for use in preventing or treating a respiratory viral infection or pneumonia caused by a viral infection. The interferon-γ is formulated in an aqueous solution at a concentration of about 0.05 to 2.5 mg of biologically active protein per mL of solution. In an embodiment, a 2.0 mL solution containing 400 μg of interferon-γ1b is administered as an aerosol produced by a breath-enhanced jet nebulizer that can produce the aerosol with a mass median aerodynamic diameter (MMAD) of about 1.0 microns. This treatment may be effective to prevent, treat, or limit the severity of the course of infection for COVID-19, influenza, and other clinically significant viral infections.

### CROSS REFERENCE TO RELATED PATENT APPLICATIONS

This patent application claims priority to U.S. patent application 63/000,469 filed Mar. 26, 2020; U.S. Patent Application 63/000,525 filed Mar. 27, 2020; and U.S. Patent Application 63/056,505 filed Jul. 24, 2020, the contents of each of which are incorporated by reference.

### SEQUENCE LISTING

[0001-1] The sequence listing filed on Mar. 5, 2024 is hereby incorporated by reference in its entirety. The sequence listing is an ASCII plain text file, the file name is “AB1057-P05-US4-SEQListing ST25.txt”, the file was created on Mar. 5, 2024, and the file size is 1512 bytes.

### FIELD OF THE INVENTION

This invention pertains to the use of cytokines, in particular interferon gamma, to treat people suffering from a viral disease, including COVID-19 and influenza.

### BACKGROUND

COVID-19 is a dangerous viral disease that emerged in late 2019 and rapidly spread worldwide. The causative agent of the current coronavirus disease (COVID-19) pandemic is severe acute respiratory syndrome coronavirus 2 (SARS-CoV-2). To date, more than 121 million cases of COVID-19 have been confirmed worldwide, and over 2.6 million have lost their lives. COVID-19 is characterized by two critical features. First the causative virus is highly contagious and appears to be transmitted by aerosols exhaled from infected persons and inhaled. Second, a significant percentage of persons who contract the disease develop a severe pneumonia that is fatal in a significant number of patients. COVID-19 also attacks other organs, including the liver, kidneys, heart and brain.

A significant factor in the danger of this disease is current treatments for COVID-19 have limited effectiveness and mortality is high. At present vaccines have recently become available, but this approach does not address potentially significant non-vaccinated populations, and potentially resistant viral variants are a concern. Accordingly, treatments are urgently needed to arrest the death rate of COVID-19 disease.

SARS-CoV-2 is adept at evading innate immunity, the naïve host's primary defense against a newly emerged coronavirus.¹ The virus employs numerous structural and non-structural proteins that inhibit interferon (IFN) production and function. Through blocking IFN, viral replication can proceed unchecked. In addition to antiviral functions, circulating IFN alerts the host to viral infection with symptoms such as fever, pain and fatigue. This capacity for stealth replication in the absence of symptoms allows asymptomatic transmission, making it particularly difficult to control from a public health perspective.

Interferons are a family of naturally occurring proteins that are produced by cells of the immune system.² Three classes of interferons have been identified, alpha, beta and gamma. Each class has different effects though their activities overlap. Together, the interferons direct the immune system's attack on viruses, bacteria, tumors and other foreign substances that may invade the body. Once interferons have detected and attacked a foreign substance, they alter it by slowing, blocking, or changing its growth or function.

Interferon-γ (IFN-γ) is a pleiotropic cytokine that has specific immune-modulating effects, e.g. activation of macrophages, enhanced release of oxygen radicals, microbial killing, enhanced expression of MHC Class II molecules, anti-viral effects, induction of the inducible nitric oxide synthase gene and release of NO, chemotactic factors to recruit and activate immune effector cells, downregulation of transferrin receptors limiting microbial access to iron necessary for survival of intracellular pathogens, etc.

Genetically engineered mice that lack interferon-γ or its receptor are extremely susceptible to mycobacterial infection. Interferon-γ has been shown to have antiviral and immunomodulatory activity.³

Human interferon gamma is a cytokine belonging to a diverse group of interferons, which have a crucial immunological function in a wide variety of viral infections. IFN-γ is synthesized and secreted by CD4+ T helper cell type 1 (Th I) lymphocytes, CDS+ cytotoxic lymphocytes and natural killer (NK) cells. As a part of the innate immune response, IFN-γ promotes antiviral immunity through its regulatory effects on the innate immune response. It acts as a key link between the innate immune response and activation of the adaptive immune response. Beyond its antiviral effects, a growing amount of evidence suggests that IFN-γ may have immunoregulatory functions that are critical for dampening immunopathogenic mechanisms and minimizing collateral damage from the infection.

A form of IFN-γ has been an approved drug in the United States since 1999. The pharmaceutical version of IFN-γ is termed “IFN-γ1b” and is a 140 amino acid sequence sold under the brand name “ACTIMMUNE®.” ACTIMMUNE is approved for the treatment of chronic granulomatous disease and osteopetrosis.⁴ ACTIMMUNE is supplied as a solution for subcutaneous injection in 0.5 mL vials containing 100 mcg of IFN-γ1b disodium succinate hexahydrate (0.37 mg), mannitol (20 mg), polysorbate 20 (0.05 mg), succinic acid (0.14 mg) and Sterile Water for Injection.⁵ The 140 amino acid sequence of IFN-γ1b in ACTIMMUNE is disclosed and claimed in U.S. Pat. No. 5,690,925 issued Nov. 25, 1997, at claim 1.⁶ The amino acid sequence of IFN-γ1b is also reported in U.S. Pat. No. 7,419,805 at SEQ ID No: 34, at col. 89, which the sequence listing and specification assert is the same sequence as the ACTIMMUNE product.

Recombinant IFN-γ was administered to normal volunteers and cancer patients in the 1980s through intramuscular and subcutaneous routes. There was evidence of monocyte activation, e.g. release of oxidants in serum from subcutaneous administration of IFN-γ, but the IFN-γ was undetectable in lung epithelial lining fluid (ELF).⁷ However, administration of IFN-γ by inhalation with a nebulizer caused dose-dependent levels of IFN-γ in ELF without being detected in serum, suggesting that inhalation of IFN-γ may be a viable route to administering this drug.⁸

Patients susceptible to intracellular infection are known to be relatively deficient in interferons. It has been shown that susceptibility to tuberculosis can be related to local interferon levels in the lungs and response to therapy is associated with local interferon synthesis.⁹ Addition of interferon by inhalation hastens clinical improvement in these patients.¹⁰ Viral infections are also mediated by cytokines in the extracellular milieu. Interferons can serve to inhibit viruses and prevent cellular infection. Administration of IFN-γ by the inhalation route has also been investigated with promising results the treatment of and idiopathic pulmonary fibrosis (IPF).^{11,12} Previous patents have disclosed the use of IFN-γ for the treatment of various other bronchial diseases, including COPD, and asthma.^{12,13} IFN-γ has been claimed for the treatment of tuberculosis.¹⁴

For COVID-19 in particular several studies have suggested that morbidity is linked to IFN-γ deficiency,¹⁵ suggesting that, like tuberculosis, the patient response to SARS-CoV-2 infection and other viruses can be enhanced by targeted therapy of IFN-γ to the lungs by inhalation.

### SUMMARY OF THE INVENTION

Embodiments of the invention described herein provide methods of preventing and treating respiratory or pulmonary viral infections using aerosolized interferon, in particular interferon-γ (IFN-γ).

In an embodiment, a method is provided for treating or preventing a respiratory viral infection or pneumonia caused by a viral infection with inhaled aerosolized interferon-γ wherein the interferon-γ is formulated in an aqueous solution at a concentration of about 0.05 to 2.5 mg of biologically active protein per mL of solution, and wherein the solution is administered as an aerosol produced by a breath-enhanced jet nebulizer that can produce the aerosol with a mass median aerodynamic diameter (MMAD) of about 1.0 microns.

In an embodiment, aerosolized interferon-γ is provided for use in treating or preventing a pulmonary viral infection or pneumonia caused by a viral infection, wherein the interferon-γ is formulated in an aqueous solution at a concentration of about 0.05 to 2.5 mg of biologically active protein per mL of solution, and wherein the solution is administered as aerosolized droplets produced by a breath-enhanced jet nebulizer that can produce the aerosol with a mass median aerodynamic diameter (MMAD) of about 1.0 microns.

In an embodiment, the viral infection is caused by a virus selected from SARS-CoV-2, SARS-CoV-1, Middle East respiratory syndrome-related coronavirus (MERS-CoV), Adenovirus, Influenza A, including subtypes, Influenza B, Parainfluenza viruses, including types 1, 2, 3, and 4, Rhinovirus/Enterovirus, Metapneumovirus, and Respiratory Syncytial Virus, including subtypes A and B.

In an embodiment, the interferon-γ is interferon-γ1b having the sequence of SEQ ID NO:1. In an embodiment, the interferon-γ is interferon-γ1b provided in a concentration of 200 μg/mL.

In an embodiment, the breath-enhanced jet nebulizer is equipped with an exhalation filter that traps exhaled droplets and aerosols to minimize exposure of surrounding people to potentially infectious agents exhaled from the patient.

In an embodiment, the interferon-γ1b is formulated in a 2 mL vial containing succinate hexahydrate in 0.10 mg to 10 mg; mannitol 5 mg to 200 mg; polysorbate 80 0.02 to 5.0 mg; succinic acid 0.050 mg to 5 mg, and sterile water for inhalation to make a 2.0 mL volume of the aqueous solution

In an embodiment, the interferon-γ1b is formulated in a 2 mL vial containing succinate hexahydrate (1.50 mg), mannitol (80 mg), polysorbate 80 (0.20 mg), succinic acid (0.55 mg) and sterile water for inhalation to make a 2.0 mL volume of the aqueous solution.

In an embodiment, the methods described above can be used to treat a viral infection caused by a coronavirus. The coronavirus may be SARS-CoV-2. In an embodiment, the methods described above can be used to treat a viral infection caused by another virus, such as an influenza virus, for example Avian Flu or Swine Flu.

In an embodiment, the IFN-γ is administered to a patient once every other day, once per day, or two to six times per day. The IFN-γ may administered to a patient for a period of from one day to 28 days. The IFN-γ may administered to a patient for a period of 14 days.

Disclosed herein is a method for treating COVID19 disease with inhaled aerosolized IFN-γ. In an embodiment, the method uses a jet nebulizer. In an embodiment, the jet nebulizer is a breath-enhanced jet nebulizer.

### DESCRIPTION OF THE DRAWINGS

FIG. 1 is a representative nebulizer apparatus.

FIG. 2 shows the bench testing apparatus.

FIG. 3A is a plot of particle distribution measured via cascade impaction. Y axis: log particle size; x axis: probability.

FIG. 3B is a plot of particle distribution as measured by cascade stage activity. Each bar corresponds to a filter in cascade stage. Y axis: % nebulizer charge; x axis: stage cutoff diameter (μm). Standard error bars are shown (n=5).

### DETAILED DESCRIPTION

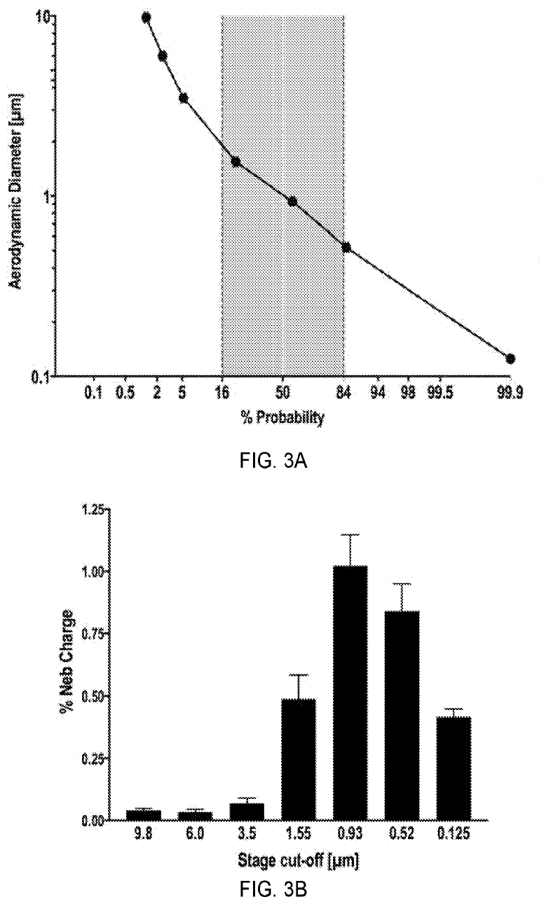

This invention is based on the putative broad spectrum antiviral activity of IFN-γ. Therefore, embodiments of the invention described herein use inhaled IFN-γ in patients presenting with bronchial viral infections caused by a viral infection, wherein the IFN-γ is administered by a jet nebulizer capable of producing an aerosol with a mass median aerodynamic diameter (MMAD) of about 1.0 μm. (See FIG. 3 for MMAD distribution data). Viral diseases that this invention may be useful in treating include COVID-19, influenza, and others.

This invention is further based on studies suggesting that interferon deficiency, in particular low levels of IFN-γ, is a factor in the pathophysiology of respiratory diseases, including tuberculosis and COVID-19.^{10, 15} The inventors postulate that IFN-γ is an important mediator in the immune response to viral diseases generally, including COVID-19, and that IFN-γ supplementation by inhalation may prevent or treat active infections. Moreover, IFN-γ supplementation in susceptible populations may prevent initial infection with viral diseases, and may prevent disease progression, for example from the upper respiratory tract to the lower respiratory tract (i.e., the lungs). Studies have documented SARS-Cov-2 replication in the upper respiratory tract via throat swabs.¹⁶ The inventors postulate that the throat may be a frequent site of initial infection, and then the disease progresses to the lungs and other tissues in the body. Thus, administration of IFN-γ according to this invention following an initial infection may prevent this progression and limit the severity of the infection.

In an embodiment, a method is provided for preventing or treating a respiratory viral infection or pneumonia caused by a viral infection with inhaled aerosolized interferon-γ wherein the interferon-γ is formulated in an aqueous solution at a concentration of about 0.05 to 2.5 mg of biologically active protein per mL of solution, and wherein the solution is administered as an aerosol produced by a breath-enhanced jet nebulizer that can produce the aerosol with a mass median aerodynamic diameter (MMAD) of about 1.0 microns. By the phrase “about,” it is meant here ±20%

In an embodiment, aerosolized interferon-γ for use in preventing or treating a pulmonary viral infection or viral pneumonia is provided, wherein the interferon-γ is formulated in an aqueous solution at a concentration of about 0.05 to 2.5 mg of biologically active protein per mL of solution, and wherein the solution is administered as aerosolized droplets produced by a breath-enhanced jet nebulizer that can produce the aerosol with a mass median aerodynamic diameter (MMAD) of about 1.0 microns.

Studies by the inventors have shown that in the breath-enhanced jet nebulizer as described herein, with an MMAD of about 1.0 microns, approximately 25% of the total protein mass in the nebulizer is actually deposited in the lungs of the patient.

In an embodiment, 2.0 mL of protein solution (the drug formulation) is added to the breath-enhanced jet nebulizer, wherein the solution contains 50 μg to 2.5 mg. In an embodiment, 2.0 mL of solution is added to the jet-nebulizer containing 200 μg/mL of solution. In an embodiment, the interferon-γ is interferon-γ1b provided in the nebulizer in dose of 100 μg to 5.0 mg. In an embodiment, the interferon-γ is interferon-γ1b provided in the nebulizer in dose of 400 μg.

In an embodiment, aerosolized interferon-γ is administered to a patient in need thereof, at a dose of 12.5 μg to 625 μg as measured delivered to the lungs as an aerosol with an MMAD of about 1.0 microns. In an embodiment, aerosolized interferon-γ is administered to a patient in need thereof, at a dose of 100 μg as measured delivered to the lungs as an aerosol with an MMAD of about 1.0 microns.

The IFN-γ for use in this invention may be IFN-γ1b. The 140 amino acid sequence of IFN-γ1b is disclosed in SEQ ID NO:1.

The IFN-γ may be administered to patients once every other day, once per day, or two to six times per day. The course of treatment may last from one day to 28 days, or the course of treatment may last for 7 days or 14 days.

### Viral Agents

Interferons generally have demonstrated antiviral activity,¹⁷ and the inventors believe that IFN-γ is likely to have activity against the following viruses that cause disease, and are transmissible via an airborne route, and infect the lungs:
SARS-CoV-2
SARS-CoV-1
Middle East respiratory syndrome-related coronavirus (MERS-CoV)
Adenovirus
Influenza A including subtypes
Influenza B
Parainfluenza viruses, including types 1, 2, 3, and 4
Rhinovirus/Enterovirus
Metapneumovirus
Respiratory Syncytial Virus, including subtypes A and B

This is not intended to be a limiting list. Other pathological viruses are likely to respond to the inventive treatment.

Many of these diseases can cause a pulmonary infection, bronchitis, or pneumonia (inflammation of the lung primarily affecting the small air sacs known as alveoli). While some of these viral diseases are more severe than others, new treatments are still very important. COVID-19, which created a worldwide pandemic in 2020, is caused by SARS-CoV-2 and there remains no effective treatment.

The viruses SARS-CoV-2, SARS-CoV-1, and MERS-CoV collectively are coronaviruses. The pathophysiology of this family of viruses varies widely. Some forms of the common cold are associated with mild coronaviruses, but SARS-CoV-2, the causative agent of COVID-19 and the 2020 pandemic is also a coronavirus.

### Nebulizers

The inventors found that specific nebulization characteristics may be critical to successful administration of interferons by the respiratory route. In particular, early studies with inhaled administration of IFN-γ were plagued by coughing, as shown by Diaz.” As discussed below, subsequent studies suggested this coughing problem was a function of the mean mass aerodynamic diameter (MMAD) of the aerosol from the nebulizer.

The inventors found that appropriate particle size of the aerosol can resolve the problem of patients coughing. In an embodiment, this invention employs a breath-enhanced jet nebulizer that controls to the MMAD to about 1.0 μm, and has a flow control orifice to control the rate of breathing and force the patient to inhale slowly and deeply. Such a nebulizer and orifice are disclosed in PCT Patent Application WO 2018/045263 A1 published Mar. 18, 2018 (the '263 publication), the contents of which are incorporated by reference.

The jet nebulizer disclosed in the '263 publication employs a Venturi and one or more baffles that draw a drug solution liquid out of a reservoir into a high-pressure air stream, where the liquid is aerosolized by shear forces and shunted to the nebulizer exit port where the nebulized drug is inhaled by a patient. An additional feature of the jet nebulizer disclosed in the '263 publication is a breath-enhancement feature, which comprises a constriction in the same area as the main Venturi that causes a secondary Venturi effect to accelerate the rate of drug delivery, by increasing the flow of drug solution to the main Venturi.

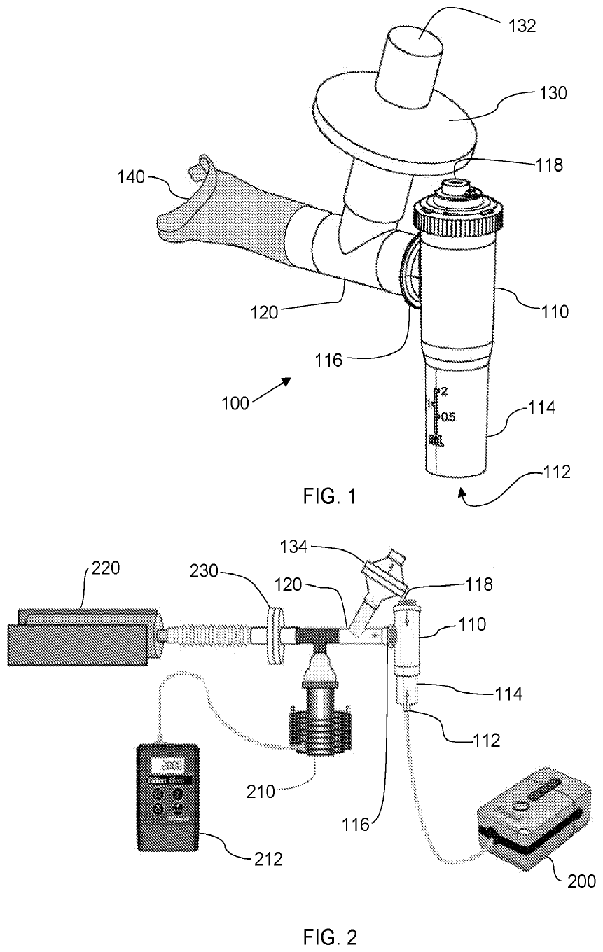

A representative nebulizer embodiment is shown in FIG. 1. This nebulizer is under commercial development as the “i-Neb Mini™” nebulizer, made by InspiRx, Inc. of Somerset, NJ. FIG. 1 shows an i-Neb Mini apparatus 100 containing a nebulizer 110, a mouthpiece 140, and various other parts. The nebulizer includes an input port 112 for pressurized air that drives the nebulization of the drug. A drug reservoir 114 integral to the nebulizer contains a solution for nebulization. Ambient air enters the nebulizer through port 118. The aerosolized drug exits the nebulizer at exit port 116 and is inhaled by a patient through mouthpiece 140. Also shown is a T-connector 120. One of the connections to T-connector 120 is an exhalation filter 130 and exhaust port 132.

The Venturi in nebulizer 100 may be powered by a portable compressor 200 at 3.4 L/min, 16 pounds per square inch (PSI), measured under load connected to the nebulizer. Inspiratory gases enter the top of the nebulizer at port 118 via a one-way valve that directs patient flow into the nebulizer for breath enhancement. In an embodiment, the nebulizer top also contains an orifice in line with the one-way valve designed to limit inspiratory flow. The diameter of the orifice was defined in separate experiments in human volunteers to provide a deep inspiration over about 6 seconds. Once inspiration begins, inspiratory flow is fixed at about 0.5 L/s no matter how much effort is used by the subject. In vitro bench studies were carried out to confirm the behavior of the nebulizer design by using this breathing pattern before testing in humans.

In an embodiment, the orifice at 118 in the nebulizer may have a diameter of from 0.50 mm to 10.0 mm. In an embodiment, the orifice has a diameter of 2.30 mm. In an embodiment, the orifice may produce a sound (such as a whistling sound) when air is flowing through it at a desired rate of flow, for example 0.5 L/s.

Five examples of the nebulizer used for human subject testing were tested on the bench by using a breathing simulator (FIG. 2). Simulated breathing used a Harvard pump, (model No. 618; Harvard Respiratory Apparatus, Millis, MA) 220 set to deliver a slow and deep sinusoidal breathing pattern (1.5 L, 6 breaths/min, duty cycle of 0.7). The long duty cycle (inspiratory time/total time of the breath) provides an average inspiratory time of about 6-7 seconds with a short expiratory time. This pattern of breathing is similar to a breath-hold, designed to maximize deposition and minimize expiratory losses. An inhalation filter 230 (Pari, Starnberg, Germany) was placed between the nebulizer and the respirator and on the expiratory port of the mouthpiece to capture all inhaled particles that would be presented to a patient (Inhaled Mass). An exhalation filter 134, was used in bench testing to trap particles generated during the expiratory phase of breathing plus particles in the tubing dead space (Expiratory Phase Filter). Particle size distribution was measured with a cascade impactor 210, a Marple 8-stage impactor, coupled to pump 212 drawing air at 2 L/min flow (purchased from Thermo Fischer Scientific, Waltham, MA) placed between the nebulizer and the breathing simulator. Percent activity per cascade stage was plotted as log aerodynamic diameter versus probability. The bench studies employed a nebulizer charge consisting of 2 mL saline mixed with 300 μCi ^{99m}Technetium bound to diethylenetriaminepentaacetic acid (DTPA).

Cascade impaction data indicated that the mass median aerodynamic diameter (geometric standard deviation) produced by the breath-enhanced nebulizer described above averaged 1.04 μm±0.03 μm.¹⁸ FIG. 3A shows a probability plot of MMAD vs. probability. FIG. 3B shows mass trapped at each stage of the cascade impactor. The greatest mass was trapped at the 0.93 μm stage. Results are skewed towards smaller particle sizes.

Note that the bench studies described in the forgoing paragraphs employ exhalation filter 134 that measures trapped particles in the dead space during an exhalation in the simulated apparatus, but in clinical use, filter 130 may be used, which traps infectious agents and aerosols in exhaled air, to limit the exposure of surrounding persons to airborne infectious particles and aerosols in the exhaled air from the patient.

Proteins may be damaged by nebulization. The i-Neb-Mini utilizes a particular low flow of high-pressure gas to generate the particles relying on the enhancement provided by the patient's own breathing. This milieu is particularly protective of the protein as shown by the full preservation of drug activity before and after nebulization.

The inventors found nebulization of ACTIMMUNE solution in the i-Neb Mini nebulizer did not result in any degradation of the IFN-γ protein by HLA-DR ELISA immunoassay.¹⁹

### Formulations

The formulation of IFN-γ in this invention may also include a buffer and a simple sugar (e.g., for protein stabilization and regulation of osmotic pressure). The buffer may be, for example, 1 N saline. The nebulizer formulation may also contain a surfactant, to reduce or prevent surface induced aggregation of the protein caused by atomization of the solution in forming the aerosol.

ACTIMMUNE IFN-γ is supplied as 100 μg in a vial containing a 0.5 mL solution containing disodium succinate hexahydrate (0.37 mg), mannitol (20 mg), polysorbate 20 (0.05 mg), succinic acid (0.14 mg) and Sterile Water for Injection. The inventors and others have used this formulation in inhalation studies, but it has shortcomings.

In an embodiment, this invention provides a pharmaceutical composition of IFN-γ1b having the amino sequence of SEQ ID NO:1. In an embodiment, the IFN-γ is formulated in an aqueous solution at a concentration of 0.05 mg to 2.5 mg per mL of solution. In an embodiment, the IFN-γ is formulated in an aqueous solution at a concentration of 200 μg/mL. In an embodiment, the IFN-γ is supplied in 2.0 mL vials that can be added to the breath-enhanced jet nebulizer of this invention, thereby providing 400 μg in the nebulizer for dosing to the patient.

Other excipients in the inventive formulation may include succinate hexahydrate in 0.05 mg to 5 mg; mannitol 2.5 mg to 100 mg; polysorbate 80 10 μg to 2.5 mg; succinic acid 25μ to 2.5 mg (all amounts in this sentence are per mL). In an embodiment, the inventive formulation comprises succinate hexahydrate (0.75 mg), mannitol (40 mg), polysorbate 80 (0.10 mg), succinic acid (0.28 mg) and sterile water for inhalation to make a 1.0 mL solution. In an embodiment, each 2.0 mL vial contains succinate hexahydrate (1.50 mg), mannitol (80 mg), polysorbate 80 (0.20 mg), succinic acid (0.56 mg) and sterile water for inhalation to make a 2.0 mL solution for packaging into vials for addition to the nebulizer.

Regarding polysorbate 80, this substance is a hydrophilic nonionic surfactant and emulsifier. Studies have found that polysorbate 80 has lower pulmonary toxicity than the polysorbate 20 used in the ACTIMMUNE subcutaneous injection formulation.²⁰

Mannitol is employed in the formulation because it was found to be necessary to stabilize the ACTIMMUNE injectable formulation.²¹ However, mannitol is also a known bronchial irritant that can cause coughing, which is undesirable in the context of administering IFN-γ by inhalation; keeping the peptide in contact with lung tissues is necessary for the desired pharmaceutical activity, and coughing may defeat that purpose. The Kanth study found that seven subjects with IPF spontaneously coughed when administered a nebulized solution of mannitol with a mass median aerodynamic diameter (MMAD) of 6.5 μm.²¹ The Kanth study also found that by appropriate breathing, using slow and deep inhalations over about 6 seconds, along with an MMAD of about 1.2 μm, no subjects coughed.²¹ Note that since the publication of the Kanth paper (n. 21), superior nebulizer technology is now available to give good flow rates with an MMAD of about 1.0 μm using the InspiRx “i-Neb Mini™” nebulizer.

### Clinical Studies

Samuel and Smaldone have published data showing that inhaled nebulized IFN-γ is effectively deposited in lung tissue.¹⁸ Bench studies showed that 41.8%±2.19% of the IFN-γ mass will enter the lungs (inhaled mass). In vivo studies with 300 μCi ^{99m}Technetium bound to diethylenetriaminepentaacetic acid (DTPA) that measured mass balance of nebulized 300 μCi ^{99m}Technetium in 2.0 mL bound to diethylenetriaminepentaacetic acid (DTPA) showed lung deposition of 25.7%±1.72% in four normal subjects, and 23.5%±1.59% in nine IPF subjects. In human subjects, inhaled mass in the lungs can be measure directly with a gamma camera. The in vivo studies show that it is possible to control drug delivery to the deep lung by using a jet nebulizer while avoiding upper airway (throat and central airways) deposition. The nebulizer mechanically controlled inspiratory time and flow, limiting inspiratory impaction and promoting deposition in small airways and alveoli via settling.

A pilot and feasibility study of inhaled IFN-γ in patients with COVID-19 infection that has not progressed to pneumonia will be performed. Approximately 200 patients will be enrolled, randomized by random block design with block sizes of 20 to placebo or inhaled IFN-γ. Subjects will be administered inhaled IFN-γ (400 μg according the formulation disclosed herein) or inhaled placebo every other day for 14 days (6 treatments). Inhaled IFN-γ will be administered at a dose of 100 and 400 μg, which will deposit 25 and 100 μg in the deep lung. That is, when 100 and 400 μg of IFN-γ is added to the nebulizer, about 25 and 100 μg respectively is actually deposited in the deep lung due to losses of aerosolized drug. Subjects will be observed for 1 hr to monitor for possible allergic reactions and pre/post spirometry performed on the day of first treatment. Following 14 days of treatment, subjects will be evaluated for short-term benefit physical exam, questionnaire and repeat nasopharyngeal sampling for COVID-19. End points such as viral load, body temperature reduction, days in hospital, energy level, use of other drugs, the need for more aggressive therapeutic interventions, etc. will be evaluated.

Sequence Listing

| | |
|---|---|
| <210> | SEQ ID NO: 1 |
| <211> | LENGTH: 140 |
| <212> | TYPE: PRT |
| <213> | ORGANISM: Artificial Sequence |
| <220> | FEATURE: |
| <223> | OTHER INFORMATION: Description of Artificial Sequence: Actimmune(r); |
| | Interferon gamma 1b |
| <400> | SEQUENCE 1 |

```
Met Gln Asp Pro Tyr Val Lys Glu Ala Glu Asn Leu Lys Lys Tyr Phe
    1 5 10 15
Asn Ala Gly His Ser Asp Val Ala Asp Asn Gly Thr Leu Phe Leu Gly
                          20 25 30
Ile Leu Lys Asn Trp Lys Glu Glu Ser Asp Arg Lys Ile Met Gln Ser
                  35 40 45
Gln Ile Val Ser Phe Tyr Phe Lys Leu Phe Lys Asn Phe Lys Asp Asp
          50 55 60
Gln Ser Ile Gln Lys Ser Val Glu Thr Ile Lys Glu Asp Met Asn Val
65 70 75 80
Lys Phe Phe Asn Ser Asn Lys Lys Lys Arg Asp Asp Phe Glu Lys Leu
                          85 90 95
Thr Asn Tyr Ser Val Thr Asp Leu Asn Val Gln Arg Lys Ala Ile His
                100 105 110
Glu Leu Ile Gln Val Met Ala Glu Leu Ser Pro Ala Ala Lys Thr Gly
        115 120 125
Lys Arg Lys Arg Ser Gln Met Leu Phe Arg Gly Arg
130 135 140
```

Drawing Legend

| | |
|---|---|
| 100 | Nebulizer apparatus for administering interferon-γ to patients with |
| | COVID-19. |
| 110 | Nebulizer |
| 112 | Nebulizer pressurized air input |
| 114 | Drug solution reservoir |
| 116 | Nebulizer output port |
| 118 | Input port with flow restricting orifice |
| 120 | T-connector |
| 130 | Exhalation filter for trapping aerosols in clinical use |
| 132 | Exhalation port |
| 134 | Exhalation filter that measures trapped particles in the dead space |
| | during an exhalation in the simulated apparatus |
| 140 | Mouthpiece |
| 200 | Air compressor |
| 210 | Cascade impactor |
| 212 | Air pump coupled to cascade impactor |
| 220 | Simulating lung pump |
| 230 | Inhalation filter on test apparatus to capture all inhaled particles |
| | that would be presented to a patient (Inhaled Mass). |

### REFERENCES

¹ Marina R. Alexander et al., “Ribosome-profiling reveals restricted post transcriptional expression of antiviral cytokines and transcription factors during SARS-CoV-2 infection,” bioRxiv 2021.03.03.433675; doi: https://doi.org/10.1101/2021.03.03.433675
² De Andrea M, et al. “The interferon system: an overview”. European Journal of Paediatric Neurology. 2002 6 Suppl A (6): A41-6, discussion A55-8. doi:10.1053/ejpn.2002.0573.
³ K. Schroder, “Interferon-γ: an overview of signals, mechanisms and functions,” Journal of Leukocyte Biology, 2004, 75: 163-189. https://doi.org/10.1189/jlb.0603252.
⁴ ACTIMMUNE® prescribing information.
⁵ Id.
⁶ See also E. Rinderknecht et al. “Natural human interferon-gamma. Complete amino acid sequence and determination of sites of glycosylation,” J. Biol. Chem. 1984, 259:6790-6797, https://doi.org/10.1016/S0021-9258(17)39797-1, and P. W. Gray, et al., “Expression of human immune interferon cDNA in E. coli and monkey cells,” Nature (1982) 295: 503-508 doi: 10.1038/295503a0.
⁷ H. A. Jaffe et al., “Organ specific cytokine therapy. Local activation of mononuclear phagocytes by delivery of an aerosol of recombinant interferon-gamma to the human lung,” J Clin Invest. 1991, 88, 297-302, doi: 10.1172/JC1115291
⁸ Id.
⁹ R. Condos et al., “Local Immune Responses Correlate with Presentation and Outcome in Tuberculosis,” Am J Respir Crit Care Med 1998, 157, 729-735, https://doi.org/10.1 164/ajrccm.157.3.9705044
¹⁰ R. Dawson, R. Condos, et al. “Immunomodulation with Recombinant Interferon-γ1b in Pulmonary Tuberculosis,” PLoS ONE 2009, 4(9), e6984. doi:10.1371/journal.pone.0006984
¹¹ K. T. Diaz et al., “Delivery and Safety of Inhaled Interferon-γ in Idiopathic Pulmonary Fibrosis,” J. Aerosol Med. Pulmonary Drug Del., 2012, 25(2), 1-9; DOI: 10.1089/jamp.2011.0919.
¹² U.S. Pat. No. 6,964,761 B1, issued Nov. 15, 2005
¹³ WO2007/035180 A1 published Mar. 29, 2007
¹⁴ U.S. Pat. No. 8,110,181 B2 issued Feb. 7, 2012
¹⁵ Z. J. Hu et al., “Lower Circulating Interferon-Gamma Is a Risk Factor for Lung Fibrosis in COVID-19 Patients.” Front. Immunol. 2020, 11:585647, doi: 10.3389/fimmu.2020.585647; C. A. Pierce et al., “Differences in immune responses to SARS-CoV-2 in children and adults are linked to clinical outcomes,” Science Translational Medicine 2020, 12(564), eabd5487, DOI: 10.1126/scitranslmed.abd5487; C. I. van der Made C I, et al. “Presence of Genetic Variants Among Young Men With Severe COVID-19,” JAMA. 2020; 324(7):663-673. doi:10.1001/jama.2020.13719.
¹⁶ Wölfel, R., Corman, V. M., Guggemos, W. et al. “Virological assessment of hospitalized patients with COVID-2019,” Nature 2020, 581, 465-469. https://doi.org/l0.1038/s41586-020-2196-x
¹⁷ See Schroder, n. 3 at 174.
¹⁸ J. Samuel and G. C. Smaldone, “Maximizing Deep Lung Deposition in Healthy and Fibrotic Subjects During Jet Nebulization,” J. Aerosol Med. Pulmonary Drug Del. 2020, 33(2), 108-115, DOI: 10.1089/jamp.2019.1552; incorporated herein by reference.
¹⁹ Watanabe, N., Suzuki, Y., Yonezu, T. et al. “A cell-based high-throughput screening assay system for inhibitor compounds of antigen presentation by HLA class II molecule.” Sci Rep 7, 6798 (2017). https://doi.org/10.1038/s41598-017-07080-4
²⁰ F. Lindenberg, et al., “Evaluation of Lung Cell Toxicity of Surfactants for Inhalation Route,” J Toxicol Risk Assess 2019, 5:022. doi.org/10.23937/2572-4061.1510022; J. K. Metz et al., “Safety Assessment of Excipients (SAFE) for Orally Inhaled Drug Products,” ALTEX 37(2), 275-286. doi:10.14573/altex.1910231
²¹ P. M. Kanth et al., “Nebulized Mannitol, Particle Distribution, and Cough in Idiopathic Pulmonary Fibrosis,” Respiratory Care August 2018, respcare.06153; DOI: 10.4187/respcare.06153

1. A method of treating or preventing a respiratory viral infection or pneumonia caused by a viral infection with inhaled aerosolized interferon-γ, wherein the interferon-γ is formulated in an aqueous solution at a concentration of about 0.05 to 2.5 mg of biologically active protein per mL of solution, and wherein the solution is administered as an aerosol produced by a breath-enhanced jet nebulizer that can produce the aerosol with a mass median aerodynamic diameter (MMAD) of about 1.0 microns.

2. Aerosolized interferon-γ for use in treating or preventing a respiratory viral infection or pneumonia caused by a viral infection, wherein the interferon-γ is formulated in an aqueous solution at a concentration of about 0.05 to 2.5 mg of biologically active protein per mL of solution, and wherein the solution is administered as aerosolized droplets produced by a breath-enhanced jet nebulizer that can produce the aerosol with a mass median aerodynamic diameter (MMAD) of about 1.0 microns.

3. The method of claim 1, wherein the viral infection is caused by a virus selected from SARS-CoV-2, SARS-CoV-1, Middle East respiratory syndrome-related coronavirus (MERS-CoV), Adenovirus, Influenza A, including subtypes, Influenza B, Parainfluenza viruses, including types 1, 2, 3, and 4, Rhinovirus/Enterovirus, Metapneumovirus, and Respiratory Syncytial Virus, including subtypes A and B.

4. The method of claim 1, wherein the interferon-γ is interferon-γ1b having the sequence of SEQ ID NO:1.

5. The method of claim 1, wherein the interferon-γ is interferon-γ1b provided in the nebulizer in a concentration of 200 μg/mL.

6. The method of claim 1, wherein the interferon-γ is interferon-γ1b provided in the nebulizer in dose of 100 μg to 5.0 mg.

7. The method of claim 1, wherein the interferon-γ is interferon-γ1b provided in the nebulizer in dose of 400 μg.

8. A method of preventing a respiratory viral infection from progressing to pneumonia according to the method of claim 1.

9. The method of claim 1 wherein an expiratory filter is provided on the breath-enhanced jet nebulizer.

10. The method of claim 1, wherein the aqueous solution comprises, in a 2 mL vial, succinate hexahydrate in 0.10 mg to 10 mg; mannitol 5 mg to 200 mg; polysorbate 80 0.02 to 5.0 mg; succinic acid 0.050 mg to 5 mg and sterile water for inhalation to make a 2.0 mL volume of the aqueous solution.

11. The method of claim 1, wherein the aqueous solution comprises, in a 2 mL vial, succinate hexahydrate (1.50 mg), mannitol (80 mg), polysorbate 80 (0.20 mg), succinic acid (0.55 mg) and sterile water for inhalation to make a 2.0 mL volume of the aqueous solution.

12. The method of treatment according to claim 1, wherein the virus is a coronavirus.

13. The method of treatment according to claim 12, wherein the coronavirus is SARS-CoV-2.

14. The method of treatment according to claim 1, wherein the virus is an influenza virus.

15. The method of treatment according to claim 14, wherein the influenza A is Avian Flu or Swine Flu.

16. The method of treatment according to claim 1, wherein the interferon-γ is administered to a patient once every other day, once per day, or two to six times per day.

17. The method of treatment according to claim 16, wherein the interferon-γ is administered to a patient for a period of from one day to 28 days.

18. The method of treatment according to claim 16, wherein the interferon-γ is administered to a patient for a period of 14 days.

19. A method of treating a pulmonary infection caused by a SARS-Cov-2 virus with inhaled aerosolized interferon-γ, wherein the interferon-γ is formulated in an aqueous solution at a concentration of about 0.05 to 2.5 mg of biologically active protein per mL of solution, and wherein the solution is administered as aerosolized droplets produced by a breath-enhanced jet nebulizer that can produce the aerosol with a mass median aerodynamic diameter (MMAD) of about 1.0 microns.
